(19) United States
(12) Patent Application Publication
(10) Pub. No.: US 2024/0404053 A1
(43) Pub. Date: Dec. 5, 2024

(54) Automatic Estimation of Ulcerative Colitis Severity from Endoscopy Videos USING ORDINAL MULTI-INSTANCE LEARNING

(71) Applicant: Janssen Research & Development, LLC, Raritan, NJ (US)

(72) Inventors: Evan Schwab, San Diego, CA (US); Kristopher Standish, San Diego, CA (US); Christel Chehoud, Allentown, PA (US); Gabriela Oana Cula, Princeton, NJ (US); Louis Roland Ghanem, Villanova, PA (US)

(21) Appl. No.: 18/693,084

(22) PCT Filed: Sep. 16, 2022
(86) PCT No.: PCT/IB2022/058774

§ 371 Date: Mar. 18, 2024

Related U.S. Application Data
(60) Provisional application No. 63/247,248, filed on Sep. 22, 2021.

(51) Int. Cl. G06T 7/00; G16H 50/20

(52) U.S. Cl. CPC G06T 7/0012; G16H 50/20; G06T 2207/10016; G06T 2207/10068; G06T 2207/20076; G06T 2207/20081; G06T 2207/20084; G06T 2207/30028

(57) ABSTRACT

An estimation system automatically estimates a severity of ulcerative colitis (UC) based on an endoscopic video. During a training phase, a training system trains one or more machine-learned models based on a set of training videos each annotated with a single video-level UC severity score representing an aggregate UC severity observed in the whole video. The one or more machine-learned models are capable of estimating UC severity depicted in an individual endoscopic video frame. Applying the one or more machine-learned models to an endoscopic test video of unknown UC severity enables estimation of frame-level UC severity scores for each frame of the test video. The frame-level UC severity scores may be represented on a continuous severity scale or may be mapped to discrete values on a predefined baseline severity scale such as a Mayo Endoscopic Subscore (MES) scale.

### CROSS-REFERENCE TO RELATED APPLICATIONS

This application claims the benefit of U.S. Provisional Application No. 63/247,248 filed on Sep. 22, 2021, which is incorporated by reference herein.

### BACKGROUND

### Technical Field

The described embodiments relate to an automated system for estimating ulcerative colitis severity based on endoscopic video frames.

### Description of the Related Art

Ulcerative colitis (UC) is a disabling and chronic inflammatory bowel disease (IBD) characterized by relapsing inflammation and ulceration of the large intestinal mucosa. Clinical trials in IBD use standardized scoring systems to assess both clinical outcomes and changes in disease activity. One disease severity score used in UC is the total Mayo score, which combines clinical disease features, physician global assessment, and mucosal disease burden as determined by video endoscopy. Endoscopic videos are commonly assessed by the Mayo Endoscopic Subscore (MES) which is used to define patient-level UC severity on the following scale: No UC (0), Mild UC (1). Moderate UC (2), Severe UC (3).

Under the generally accepted scoring system, gastroenterologists attribute a single MES to a video based upon the maximum disease severity observed in the video. For example, if a single video frame consists of severe UC, and the remainder of the colon is normal, then the entire video is reported with an MES=3. Therefore, a patient with severe UC spread throughout the large intestine will have the same MES as a patient with severity in only one location. The difficulty of accurately assessing UC severity using convention techniques is further complicated by the highly subjective nature of manual scoring and the lack of granularity in the conventional MES scale.

### BRIEF DESCRIPTION OF THE DRAWINGS

FIG. 1 is an example embodiment of a UC severity estimation system.

FIG. 2 is an example embodiment of a learning system for training a set of machine-learned binary classification models in a UC severity estimation system.

FIG. 3 is an example embodiment of a scoring system for scoring an input endoscopic video for UC severity based on a set of machine-learned binary classification models.

FIG. 4A is a first example embodiment of a process for combining a set of binary probabilities to generate a frame-level UC severity score for an input frame.

FIG. 4B is a second example embodiment of a process for combining a set of binary probabilities to generate a frame-level UC severity score for an input frame.

FIG. 4C is a third example embodiment of a process for combining a set of binary probabilities to generate a frame-level UC severity score for an input frame.

FIG. 5 is an example of a plot showing frame-level continuous UC severity scores and corresponding values on an MES scale.

FIG. 6 is an example embodiment of a process for automatically estimating UC severity from an input endoscopic video.

FIG. 7 is an example embodiment of a regression-based learning system for training a regression-based machine-learned model in a UC severity estimation system.

FIG. 8 is an example embodiment of a scoring system for scoring an input endoscopic video for UC severity based on a regression-based machine-learned model.

FIG. 9 is an example embodiment of a process for automatically estimating UC severity from an input endoscopic video using a regression-based machine-learned model.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

An estimation system automatically estimates a severity of ulcerative colitis (UC) based on an endoscopic video. During a training phase, a training system trains one or more machine-learned models based on a set of training videos each annotated with a single video-level UC severity score representing an aggregate UC severity observed in the whole video. The one or more machine-learned models are capable of estimating UC severity depicted in an individual endoscopic video frame. Applying the one or more machine-learned models to an endoscopic test video of unknown UC severity enables estimation of frame-level UC severity scores for each frame of the test video. The frame-level UC severity scores may be represented on a continuous severity scale or may be mapped to discrete values on a predefined baseline severity scale such as a Mayo Endoscopic Subscore (MES) scale.

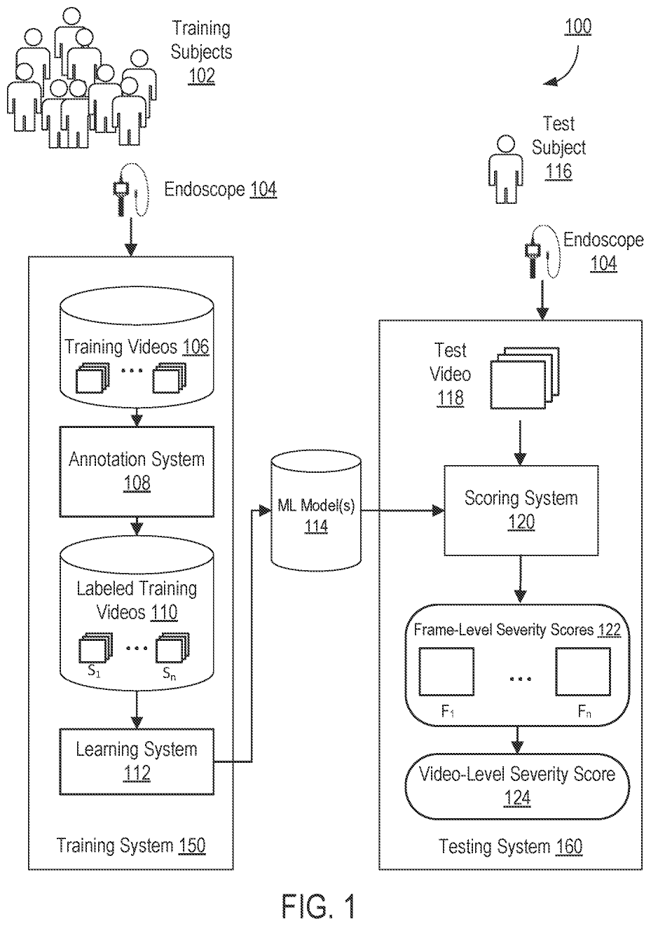

FIG. 1 illustrates an example embodiment of a UC severity estimation system 100. The UC severity estimation system 100 applies a machine learning approach in which a training system 150 learns one or more machine-learned models 114 that are applied by a testing system 160 to automatically generate frame-level severity scores 122 estimating UC severity in respective frames of an endoscopic video 118. Optionally, the estimation system 100 also automatically computes a video-level score 124 from the frame-level scores 122 that estimates an overall UC severity observed in the endoscopic video 118. The automatically generated frame-level scores 122 provide a more precise assessment of disease distribution and severity in UC than a conventional manually assessed video-level score. These frame-level scores 122 beneficially provide measures of disease activity with a broader dynamic range than a manually generated video-level score and can allow for finer assessments of meaningful therapeutic effects in UC clinical trials. Furthermore, the automatically generated frame-level scores 122 eliminate the human subjectivity inherent in manually assessed UC severity scores.

The training system 150 learns one or more machine-learned models 114 based on a set of training videos 106 obtained from a set of training subjects 102. The training videos 106 each comprise a sequence of frames captured by an endoscope 104 as it traverses through the colon of a training subject 102. Thus, different frames of each training video 106 may represent different cross-sections of the colon and may depict varying levels of UC severity present in different regions of the colon. The set of training subjects 102 may have varying levels of UC that present differently in different training subjects 102. Generally, the number of training subjects 102 and variations in UC severity are sufficiently representative of the general population to enable a robust machine-learning approach from the set of training videos 106.

The training system 150 includes an annotation system 108 and a learning system 112. The annotation system 108 obtains a single label for each of the training videos 106 and outputs a set of labeled training videos 110 having respective labels S₁, . . . , Sₙ. Here, each label represents a score for the corresponding labeled training video 110 according to a predefined baseline severity scale. The score for the labeled training video 110 may comprise a single value representing an aggregation of the varying levels of UC severity observed in the labeled training video 110. For example, the aggregation may comprise a maximum function that outputs a score indicative of the maximum (i.e., most severe) observed UC severity in the training video 110. For annotation purposes, the UC severities may be manually assessed (e.g., by a gastroenterologist or other expert) according to a set of scoring guidelines associated with the baseline severity scale. In an example embodiment, the baseline severity scale comprises an MES scale. In this case, each of the training videos 106 is labeled with a discrete severity score of 0, 1, 2, or 3 representing the maximum UC severity observed in the training video 106. In alternative embodiments, a different severity scale may be used that may have a different range, different level of granularity, and/or different scoring guidelines.

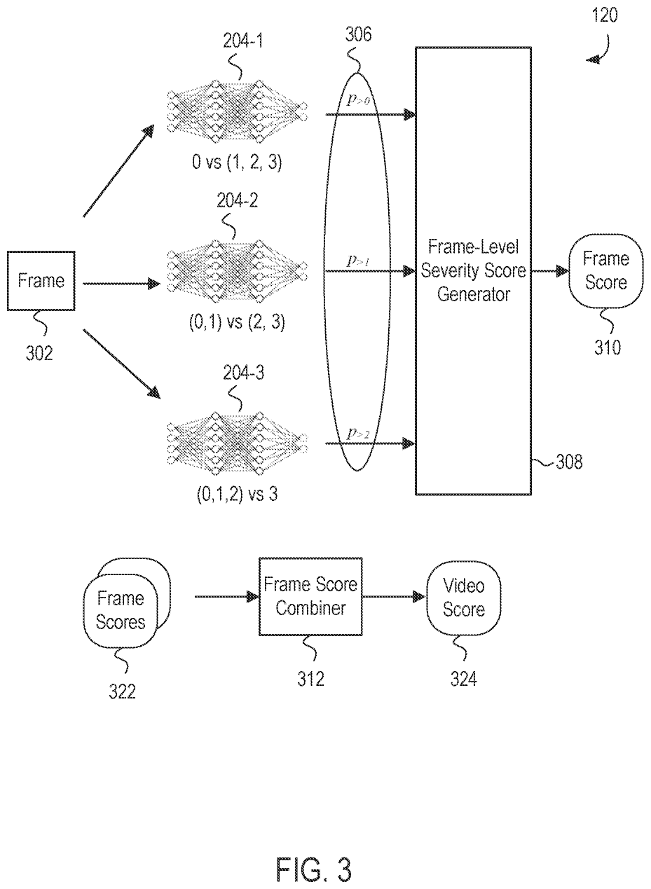

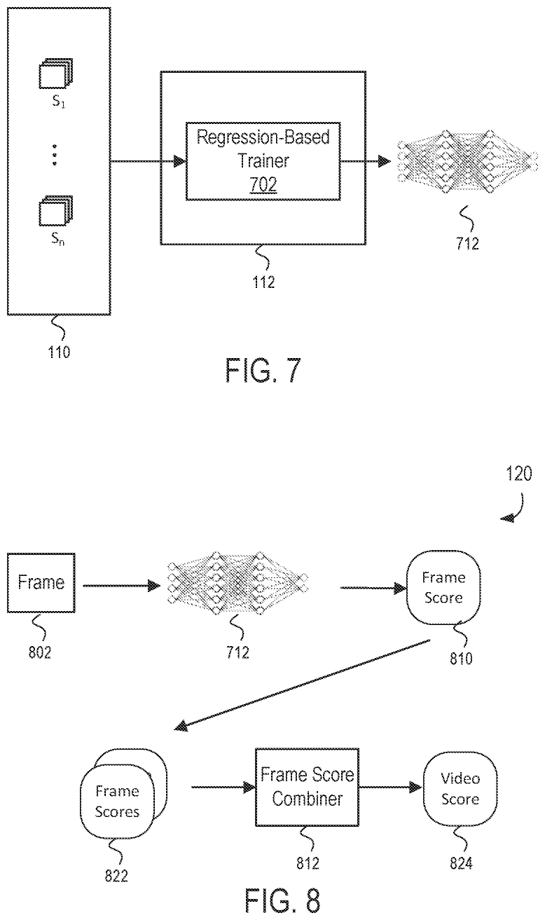

The learning system 112 generates one or more machine learned models 114 from the labeled training videos 110 using a machine-learning technique. In an embodiment, the learning system 112 solves a weakly labeled problem in which each labeled data set (i.e., a labeled training video 110) is viewed as a collection of smaller un-labeled instances (i.e., individual frames of each video 110). Utilizing the annotated video-level scores as the only input labels, the learning system 112 trains the one or more machine-learned models 114 to learn relationships between image features of an individual endoscopic video frame and the severity scores that were attributed to videos 110 containing frames having those features. Thus, the trained machine-learned models 114 can predict a UC severity score for an individual video frame even though the input labels only provide a video-level score (i.e., frame-level labels are not available for the training set 110). An example of a training methodology that operates in this framework is Multi-Instance Learning (MIL). The machine-learned models 114 may comprise, for example, convolutional neural networks (CNNs), other types of neural networks, or different types of machine-learned models capable of achieving the functions described herein. Examples embodiments of learning systems 112 using this approach are described in further detail below with respect to FIGS. 3 and 7.

In an alternative embodiment, the learning system 112 may obtain frame-level labels for at least some of the individual video frames of the training videos 106. In this case, the learning system 112 may apply a supervised (or a semi-supervised) learning approach that does not necessarily follow the MIL framework. For example, a supervised learning approach can directly learn correlations between features of individually labeled video frames of and their respective labels.

The testing system 160 includes a scoring system 120 that applies the machine-learned model(s) 114 to an input test video 118 captured by an endoscope 104 from a test subject 116. Here, the UC severity of the test subject 116 is initially unknown and the test video 118 is unlabeled. The testing system 160 generates a frame-level severity score (Fₗ, . . . , Fₙ) 122 for each frame of the test video 118 based on application of the one or more machine-learned models 114.

The frame-level severity scores 122 may comprise either continuous scores that fall within a continuous range of possible scores or discrete scores that are selected from the set of discrete values of the baseline severity scale (e.g., the MES scale). The continuous range of a continuous frame-level severity score may correspond to the same range as the baseline severity scale used in training. For example, a continuous frame-level severity scores 122 corresponding to the MES scale may comprise any value in the range [0, 3]. Here, integer values of the continuous frame-level severity scores 122 approximately correlate to the level of UC severity represented by the corresponding discrete values on the MES scale. Decimal values of the continuous frame-level severity score 122 approximate UC severity levels in between the discrete severity levels on the MES scale. For example, a continuous frame-level severity score of 2.5 signifies an approximate UC severity level in between 2 and 3 on the MES scale. Thus, a continuous frame-level severity score can provide increased granularity relative to a scale based on discrete values, such as the MES scale.

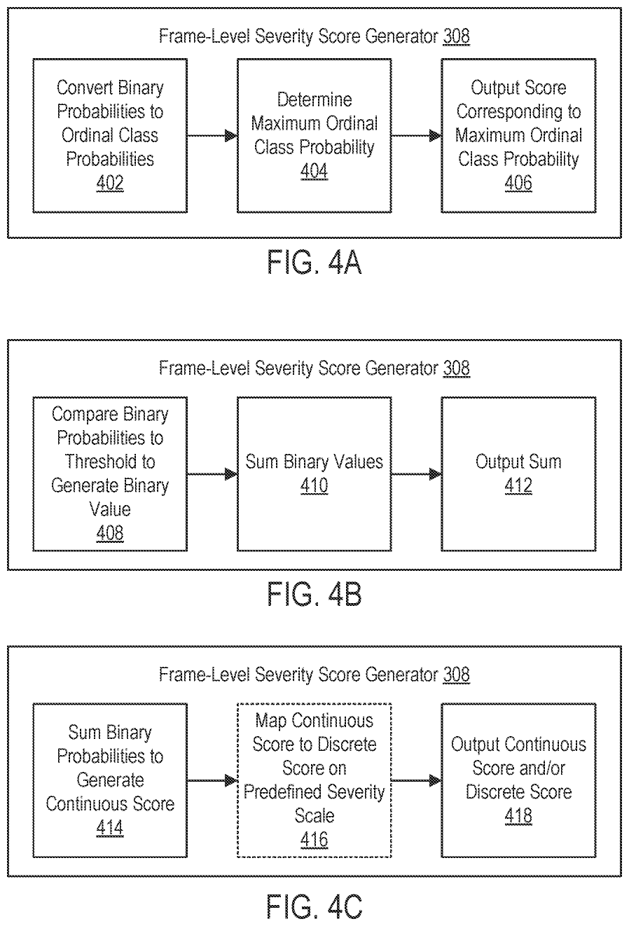

The scoring system 120 may optionally combine the set of frame-level severity scores 122 for frames of a test video 118 to generate a video-level severity score 124. For example, for consistency with the MES scale, the scoring system 120 may output a video-level severity score 124 as a discrete value based on the maximum observed frame-level severity score 122 in the test video 118. In alternative embodiments, the frame-level severity scores 122 and/or the video-level severity score 124 may be based on a different severity scale that has a different range of values or has a different level of granularity than the baseline severity scale applied to the labeled training videos 110. Example embodiments of a scoring system 120 are described in further detail below with respect to FIGS. 4 and 8.

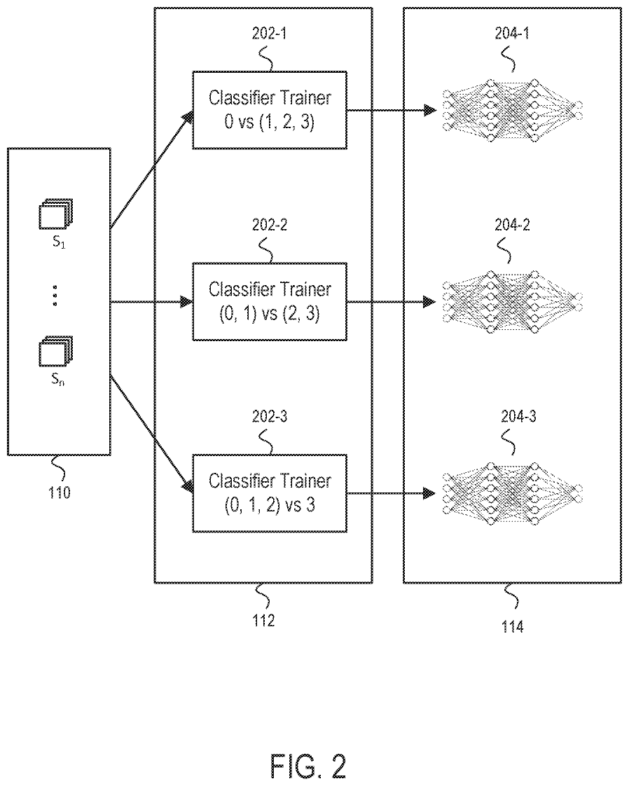

FIG. 2 illustrates an example embodiment of a learning system 112. In this embodiment, the learning system 112 comprises a set of classifier trainers 202 (e.g., classifier trainers 202-1, 202-2, 202-3) that are each associated with a different severity score threshold of the baseline severity scale applied to the training videos 110. Each classifier trainer 202 separately trains a corresponding binary classifier 204 (e.g., binary classifiers 204-1, 204-2, 204-3) to map an input video frame to a binary probability that represents a likelihood of the UC severity depicted in that input video frame being greater than the configured threshold for that classifier 204. For example, in an estimation system 100 based on the MES scale, the learning system 112 may comprise three classifier trainers 202 that train three respective binary classifiers 204: (1) a first classifier trainer 202-1 that trains a first binary classifier 204-1 to estimate a probability p>0 of the UC severity score being greater than 0 (i.e., the binary classifier 204-1 estimates the likelihood of a video frame having a score in the set {1, 2, 3}); (2) a second classifier trainer 202-2 that trains a second binary classifier 204-2 to estimate the probability p>1 of the UC severity score being greater than 1 (i.e., the binary classifier 204-2 estimates the likelihood of a video frame having a score in the set {2, 3}; and (3) a third classifier trainer 202-3 that trains a third binary classifier 204-3 to estimate the probability p>2 of the UC severity score being greater than 2 (i.e., the binary classifier 204-3 estimates the likelihood of a video frame having a score of 3). In an alternative embodiment, only two classifier trainers 202-1, 202-2 are used to train the two binary classifiers 204-1, 204-2 (i.e., the third training classifier trainer 202-3 may be omitted). Here, the output of the first binary classifier 204-1 is sufficient to detect the presence of UC and the output of the second binary classifier 204-2 is clinically useful to detect UC healing when observed over time. In alternative embodiments that use a different severity scale, a different number of classifier trainers 202 may be employed to generate a corresponding number of binary classifiers 204 according to the same approach. For example, if UC severity scale of 1-10 is used, a set of up to 9 binary classifiers may be used.

FIG. 3 illustrates an example embodiment of a scoring system 120 that operates based on a set of binary classifiers 204 having the characteristics described above. The scoring system 120 obtains the set of binary classifiers 204 and applies each of them to an individual frame 302 of an endoscopic video to obtain a set of binary probabilities 306. Here, each of the binary probabilities 306 represents a likelihood that the frame 302 depicts a UC severity above classification threshold associated with the corresponding binary classifier 204. For example, using the MES scale, a first binary probability p>0 represents the likelihood that the UC severity is greater than 0 (i.e., 1, 2 or 3 on the MES scale), a second binary probability p>1 represents the likelihood that the severity is greater than 1 (i.e., 2 or 3 on the MES scale), and the third binary probability p>2 represents a likelihood that the severity score is greater than 2 (i.e., 3 on the MES scale). In an embodiment, the binary probabilities p>0, p>1, p>2 are in the range [0, 1].

The frame-level severity score generator 308 combines the set of binary probabilities 306 for the frame 302 to generate a frame-level severity score 310. Here, the frame-level severity score generator 308 converts the binary probabilities to an ordinal score representing the level of UC severity. The frame-level severity score 310 can be selected from the discrete values of the baseline severity scale (e.g., 0, 1, 2, or 3 from the MES scale) or may be computed as a continuous frame-level severity score. Optionally, the frame-level severity score generator 308 outputs both a continuous frame-level severity score and the closest matching discrete frame-level severity score selected from the baseline severity scale.

The scoring system 120 may also include a frame score combiner 312 that combines a set of frame-level severity scores 322 for a test video 118 to generate a video-level severity score 324 attributable to the whole video 118. For example, the frame score combiner 312 may select the maximum observed frame-level severity score as the video-level severity score 324. Alternatively, the frame score combiner 312 may apply a different aggregation function (e.g., a median or averaging function) to generate the video-level severity score 324. If the frame-level severity scores 322 are continuous scores, the frame score combiner 312 may combine the continuous frame-level severity scores 322 in a manner that generates a video-level severity score 324 as a discrete value on the baseline severity scale.

FIGS. 4A-C illustrate three alternative example embodiments of processes that may be performed by the frame-level severity score generator 308 to generate the frame-level severity 310 from the set of binary probabilities 306. In a first example technique of FIG. 4A, the frame-level severity score generator 308 first converts 402 the binary probabilities 306 to a set of ordinal class probabilities. Each of the ordinal class probabilities represents a probability that the test frame 302 most closely corresponds to a specific discrete value on the baseline severity scale. For example, the binary probabilities 306 may be converted to a set of four ordinal class probabilities that respectively represent probabilities of the test frame 302 most closely corresponding to 0, 1, 2, and 3 on the MES scale. For example, a set of ordinal class probabilities p may be computed as follows: p₀=1−p_{>0}; p₁−p_{>0}−p_{>1}; p₂=p_{>1}−p_{>2}; p3=p_{>2}. The frame-level severity score generator 308 then identifies 404 the maximum probability from the set of ordinal class probabilities and outputs 406 the discrete score (e.g., 0, 1, 2, or 3) having the highest probability as the frame-level severity score 310. In other words, the frame-level severity score generator 308 selects the discrete value from the baseline severity scale that provides the best estimate of the observed UC severity level.

In a second example technique of FIG. 4B, the frame-level severity score generator 308 first compares each of the binary probabilities 306 to a threshold (e.g., 0.5) and outputs a binary value representing the comparison result (e.g., 0 or 1). The frame-level severity score generator 308 then sums 410 the set of binary values and outputs 412 the sum as the frame-level severity score 310. This technique results in discrete severity scores corresponding to the baseline severity scale (e.g., 0, 1, 2, or 3 on the MES scale).

In a third example technique of FIG. 4C, the frame-level severity score generator 308 first sums 414 the binary probabilities to generate a continuous frame-level severity score (e.g., in the range [0, 3]). The frame-level severity score generator 308 optionally maps 416 the continuous frame-level severity score to a discrete continuous frame-level severity score based on a set of threshold comparisons. For example, the frame-level severity score generator 308 may round the continuous frame-level severity score to the nearest discrete value on the baseline severity scale. The frame-level severity score generator 308 then outputs 418 either the continuous frame-level severity score, the discrete frame-level severity score, or both as the frame-level severity score 310 for the test frame 302.

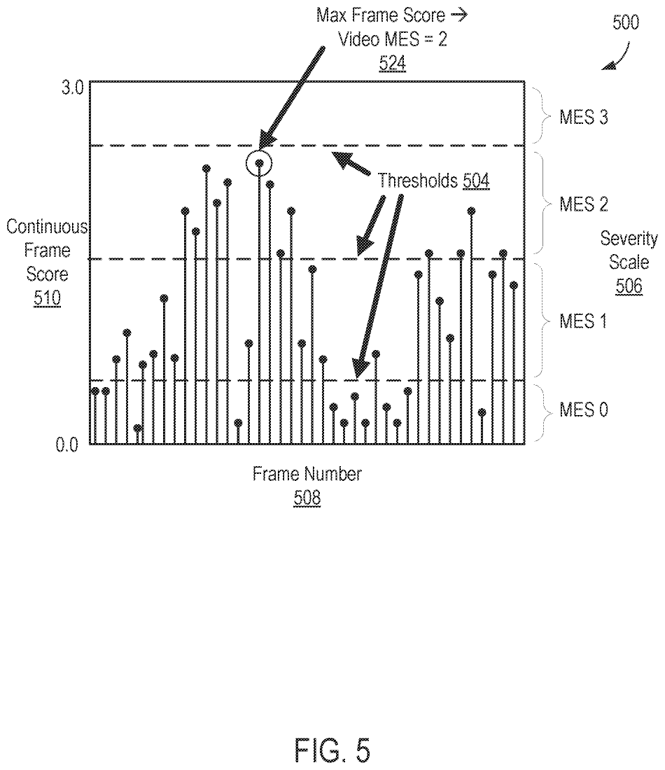

FIG. 5 is a plot 500 illustrating example data for a set of continuous frame-level severity scores 510 derived from a test video 118 having a set of frames (identified by corresponding frame numbers 508). In this example, the continuous frame-level severity scores 510 are computed in the range [0, 3] corresponding to the range of the MES scale. Each of the continuous frame-level severity scores 510 are compared to a set of thresholds 504 to bin the continuous frame-level severity scores 510 into one of a set of ranges that each approximately correlate to a discrete value on the baseline severity scale 506. A video-level severity score 524 is also estimated based on the maximum observed frame-level score. In this example, a video score of MES=2 is estimated.

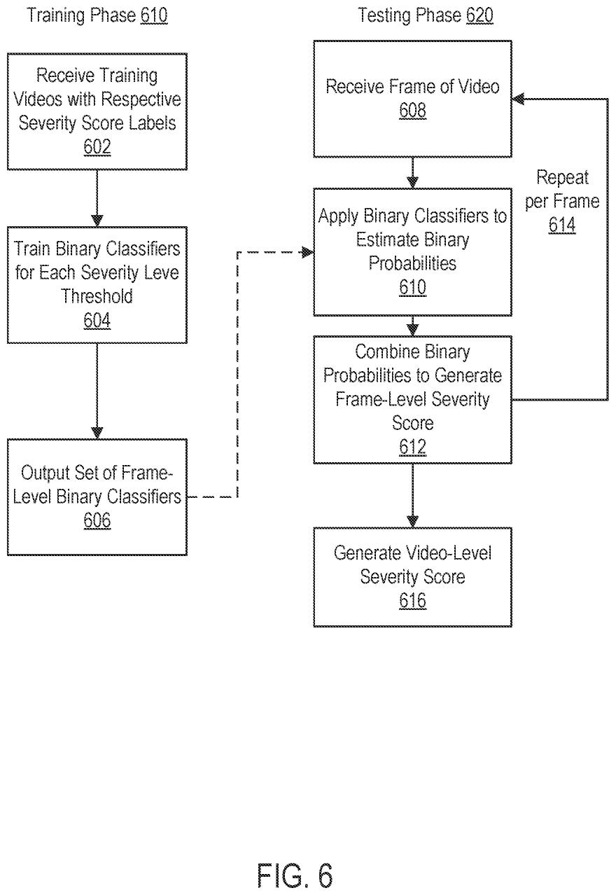

FIG. 6 is an example embodiment of a process for automatically estimating a UC severity score for an endoscopic video. In a training phase 610, an estimation system 100 receives 602 a set of training videos that are labeled with respective discrete severity scores from a baseline severity scale. The estimation system 100 trains 604 a set of binary classifiers. Each binary classifier generates a probability of a frame depicting a UC severity of a respective threshold level on the baseline severity scale as described above. The estimation system 100 outputs 606 the set of binary classifiers. In the testing phase 620, the estimation system 100 receives 608 a frame of an endoscopic video and applies 610 the set of binary classifiers to estimate respective binary probabilities associated with the different thresholds. The estimation system 100 combines 612 the binary probabilities to generate a frame-level severity score on an ordinal scale. The process repeats 614 for each frame to generate a set of independent frame-level severity scores for the test video. The estimation system 100 may then generate 616 a video-level severity score for the test video based on the set of frame-level severity scores.

FIG. 7-9 illustrate an alternative embodiment of an estimation system 100 and corresponding process that uses a regression-based machine learning technique instead of relying on binary classifiers. FIG. 7 illustrates an alternative example embodiment of a learning system 112. In this embodiment, the learning system 112 comprises a regression-based trainer 702 that trains a regression-based machine-learned model 712 based on the labeled set of training videos 110. The regression-based machine-learned model 712 is trained using regression-based techniques to output a frame-level continuous severity score in the same range as the baseline severity scale (e.g., [0, 3]). The regression-based machine-learned model 712 may comprise a CNN, a different type of neural network, or a different type of machine-learned model that is capable of achieving the functions described herein.

FIG. 8 illustrates an alternative example embodiment of a scoring system 120 utilizing the regression-based machine-learned model 712. Here, the scoring system 120 receives a test frame 802 and applies the regression-based machine-learned model 812 to generate a continuous frame-level severity score 810. A set of continuous frame-level severity scores 822 for a test video 118 may then be combined by a frame score combiner 812 to generate a video-level severity score 824 in the same manner described above.

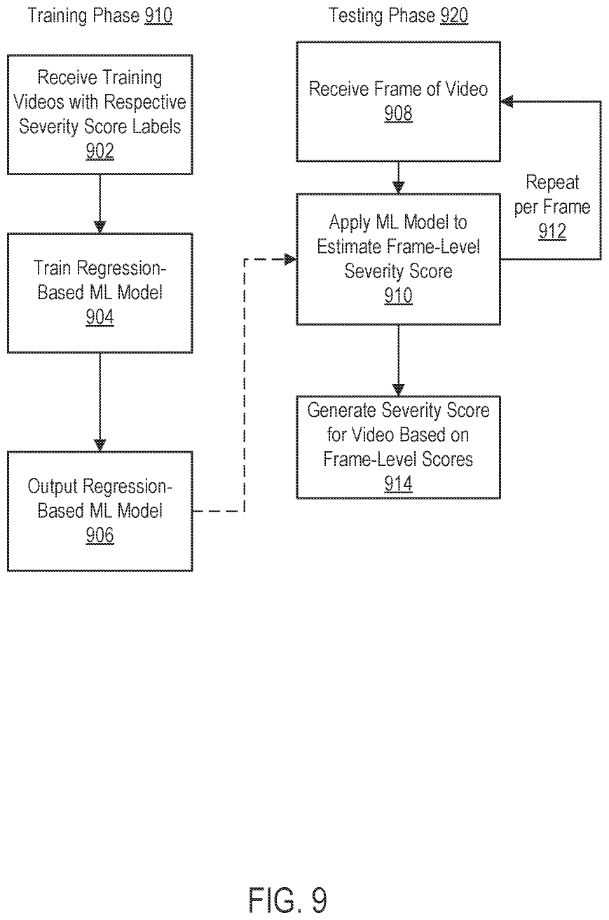

FIG. 9 is a flowchart illustrating an example embodiment of a process for automatically estimating a UC severity score for an endoscopic video using a regression-based machine-learning approach. In a training phase 910, the estimation system 100 receives 902 a set of training videos that are labeled with respective discrete severity scores from a baseline severity scale. The estimation system 100 trains 904 a regression-based machine-learned model and outputs 906 the model for use in testing. In the testing phase 920, the estimation system 100 receives 908 a frame of an endoscopic video and applies 910 the regression-based machine-learned model to estimate a frame-level continuous severity score. The process repeats 912 for each frame to generate a set of independent frame-level severity scores for the test video. The estimation system 100 then generates 914 a video-level severity score for the test video based on the set of frame-level scores in the same manner described above.

In various embodiments, the frame-level severity scores and/or the video-level severity score may be presented in a user interface according to various presentation techniques. For example, in one embodiment, a user interface available to a health care provider or patient may present a plot similar to FIG. 5 that indicates a set of frame-level severity scores for a video (continuous and/or discrete) and/or a video-level severity score. In a further embodiment, the frame-level severity scores and their corresponding video score may be stored as metadata in association with frames of an endoscopic video. Here, a user interface may display corresponding frame-level severity scores in a frame-by-frame manner during playback of a stored endoscopic video. In another embodiment, a plot of frame-level severity scores may be generated and displayed in substantially real-time as an endoscopic video is being captured. For example, the frame-level severity scores and an overall video-level severity score may be overlaid or displayed side-by-side with frames of the endoscopic video as it is being captured.

In alternative embodiments, the techniques described herein for assessing UC severity can be applied to different types of input data instead of, or in addition to, endoscopic videos. Here, the ML models can be trained on traditional images, computed tomography (CT) images, x-ray images, or other types of medical images. For example, a single label may be associated with a volumetric image and the learning system 112 is trained to estimate predictions for individual slices on the volume. The scoring system 120 can then operate on corresponding types of inputs obtained from a test subject 116 to generate severity scores 122, 124 in the same manner described above. In other embodiments, the input data may include other types of temporal signals represent sensed conditions associated with UC that are not necessarily image-based (e.g., sensor data collected over time). Here, a single label may be assigned to the signal and the machine learning system 112 is trained to estimate predictions associated with different time-limited portions.

The techniques described herein may also be employed to detect severity of other types of diseases besides UC. For example, the same techniques may be useful to detect inflammatory bowel disease (IBD) more generally, based on endoscopic video or based on other input data described above. Similar techniques may also be applied to detect severity of diseases unrelated to IBD based on relevant input videos or other input data depicting conditions indicative of severity levels. For example, such techniques may be used to detect severity of viral or bacterial infections, neurological diseases, or cardiac diseases.

Embodiments of the described estimation system 100 and corresponding processes may be implemented by one or more computing systems. The one or more computing systems include at least one processor and a non-transitory computer-readable storage medium storing instructions executable by the at least one processor for carrying out the processes and functions described herein. The computing system may include distributed network-based computing systems in which functions described herein are not necessarily executed on a single physical device. For example, some implementations may utilize cloud processing and storage technologies, virtual machines, or other technologies.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible non-transitory computer readable storage medium or any type of media suitable for storing electronic instructions, and coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

1. A method for estimating ulcerative colitis severity depicted in a frame of an endoscopic video, the method comprising:
receiving the frame of the endoscopic video;
applying a first machine-learned model to the frame of the endoscopic video to estimate a first binary probability that the frame is indicative of ulcerative colitis of greater than a first severity level on a baseline severity scale, wherein the first machine-learned model is trained from a set of annotated training endoscopic videos, and wherein each of the set of annotated training endoscopic videos has a respective single label representing a maximum severity of ulcerative colitis observed with respect to the baseline severity scale;
applying a second machine-learned model to the frame of the endoscopic video to estimate a second binary probability that the frame is indicative of ulcerative colitis greater than a second severity level on the baseline severity scale, the second severity level indicative of more severe ulcerative colitis than the first severity level, wherein the second machine-learned model is trained from the set of annotated training endoscopic videos;
generating an output severity score for the frame based on at least the first binary probability and the second binary probability; and
outputting the output severity score for the frame of the endoscopic video.

2. The method of claim 1, further comprising:
applying a third machine-learned model to the frame of the endoscopic video to estimate a third binary probability that the frame is indicative of ulcerative colitis greater than a third severity level on the baseline severity scale, the third severity level being indicative of more severe ulcerative colitis than the second severity level, wherein the third machine-learned model is trained from the set of annotated training endoscopic videos; and
wherein generating the output severity score is further based on the third binary probability.

3. The method of claim 1, wherein generating the output severity score comprises:
applying a mapping function to at least the first binary probability and the second binary probability to generate respective ordinal class probabilities for a set of discrete severity levels of the baseline severity scale; and
selecting from the set of discrete severity levels, the output severity score that corresponds to a maximum of the ordinal class probabilities.

4. The method of claim 1, wherein generating the output severity score comprises:
comparing the first binary probability to a threshold to generate a first binary value;
comparing the second binary probability to the threshold to generate a second binary value; and
determining the output severity score as a combination of at least the first binary value and the second binary value.

5. The method of claim 1, wherein generating the output severity score comprises:
combining at least the first and second binary probabilities to generate a continuous severity score;
comparing the continuous severity score to a set of thresholds to map the continuous severity score to a discrete severity level of the baseline severity scale; and
outputting the discrete severity level as the output severity score.

6. The method of claim 1, wherein generating the output severity score comprises:
combining at least the first and second binary probabilities to generate the output severity score as a continuous severity score.

7. The method of claim 1, further comprising:
storing the output severity score as an entry in a set of frame-level severity scores for the endoscopic video;
determining a maximum severity score from the set of frame-level severity scores; and
outputting the maximum severity score for the endoscopic video.

8. The method of claim 1, wherein the first machine-learned model and the second machine-learned model are each trained using a multi-instance learning algorithm.

9. The method of claim 1, wherein the baseline severity scale comprises a Mayo Endoscopic Subscore (MES) scale having discrete integer severity levels ranging from 0 to 3.

10. A non-transitory computer-readable storage medium storing instructions for estimating ulcerative colitis severity depicted in a frame of an endoscopic video, the instructions when executed causing one or more processors to perform steps comprising:
receiving the frame of the endoscopic video;
applying a first machine-learned model to the frame of the endoscopic video to estimate a first binary probability that the frame is indicative of ulcerative colitis greater than a first severity level on a baseline severity scale, wherein the first machine-learned model is trained from a set of annotated training endoscopic videos, and wherein each of the set of annotated training endoscopic videos has a respective single label representing a maximum severity of ulcerative colitis observed with respect to the baseline severity scale;
applying a second machine-learned model to the frame of the endoscopic video to estimate a second binary probability that the frame is indicative of ulcerative colitis greater than a second severity level on the baseline severity scale, the second severity level indicative of more severe ulcerative colitis than the first severity level, wherein the second machine-learned model is trained from the set of annotated training endoscopic videos;
generating an output severity score for the frame based on at least the first binary probability and the second binary probability; and
outputting the output severity score for the frame of the endoscopic video.

11. The non-transitory computer-readable storage medium of claim 10, the instructions when executed further causing the one or more processors to performs steps comprising:
applying a third machine-learned model to the frame of the endoscopic video to estimate a third binary probability that the frame is indicative of ulcerative colitis greater than a third severity level on the baseline severity scale, the third severity level being indicative of more severe ulcerative colitis than the second severity level, wherein the third machine-learned model is trained from the set of annotated training endoscopic videos; and
wherein generating the output severity score is further based on the third binary probability.

12. The non-transitory computer-readable storage of claim 10, wherein generating the output severity score comprises:
applying a mapping function to at least the first binary probability and the second binary probability to generate respective ordinal class probabilities for a set of discrete severity levels of the baseline severity scale; and
selecting from the set of discrete severity levels, the output severity score that corresponds to a maximum of the ordinal class probabilities.

13. The non-transitory computer-readable storage of claim 10, wherein generating the output severity score comprises:
comparing the first binary probability to a threshold to generate a first binary value;
comparing the second binary probability to the threshold to generate a second binary value; and
determining the output severity score as a combination of at least the first binary value and the second binary value.

14. The non-transitory computer-readable storage of claim 10, wherein generating the output severity score comprises:
combining at least the first and second binary probabilities to generate a continuous severity score;
comparing the continuous severity score to a set of thresholds to map the continuous severity score to a discrete severity level of the baseline severity scale; and
outputting the discrete severity level as the output severity score.

15. The non-transitory computer-readable storage of claim 10, wherein generating the output severity score comprises:
combining at least the first and second binary probabilities to generate the output severity score as a continuous severity score.

16. The non-transitory computer-readable storage of claim 10, wherein the instructions when executed further cause the one or more processors to performs steps comprising:
storing the output severity score as an entry in a set of frame-level severity scores for the endoscopic video;
determining a maximum severity score from the set of frame-level severity scores; and
outputting the maximum severity score for the endoscopic video.

17. The non-transitory computer-readable storage of claim 10, wherein the first machine-learned model and the second machine-learned model are each trained using a multi-instance learning algorithm.

18. The non-transitory computer-readable storage of claim 10, wherein the baseline severity scale comprises a Mayo Endoscopic Subscore (MES) scale having discrete integer severity levels ranging from 0 to 3.

19. A method for estimating ulcerative colitis severity depicted in an endoscopic video, the method comprising:
receiving the endoscopic video;
applying a regression-based machine-learned model to each frame of the endoscopic video to estimate respective frame-level severity scores representing estimated severities of ulcerative colitis in each frame, wherein the machine-learned model is trained from a set of annotated training endoscopic videos, and wherein each of the set of annotated training endoscopic videos has a respective single label representing a maximum severity of ulcerative colitis observed with respect to a baseline severity scale comprising an ordinal set of discrete severity levels;
determining a maximum frame-level severity score from the respective frame-level severity scores;
comparing the maximum frame-level severity score to a set of thresholds to select a discrete severity level from the baseline severity scale; and
outputting the discrete severity level.

20. The method of claim 19, wherein the regression-based machine-learned model is trained using a multi-instance learning algorithm.
